(19) United States
(12) Patent Application Publication
(10) Pub. No.: US 2013/0171254 A1
(43) Pub. Date: Jul. 4, 2013

(54) FAST DISSOLVING PHARMACEUTICAL COMPOSITION COMPRISING LORNOXICAM

(71) Applicants: Sanjay Boldhane, Mumbai (IN); Kuldeep Bhokare, Mumbai (IN); Shripad Jathar, Mumbai (IN); Geraldine Ann Elliott, Jindalee (AU)

(72) Inventors: Sanjay Boldhane, Mumbai (IN); Kuldeep Bhokare, Mumbai (IN); Shripad Jathar, Mumbai (IN); Geraldine Ann Elliott, Jindalee (AU)

(73) Assignee: ABBOTT HEALTHCARE PRIVATE LIMITED, Mumbai (IN)

(21) Appl. No.: 13/518,746

(22) PCT Filed: Dec. 21, 2010
(86) PCT No.: PCT/IN10/00832

§ 371 Date: Sep. 12, 2012

(30) Foreign Application Priority Data
Dec. 24, 2009 (IN) 2993/MUM/2009

(51) Int. Cl. A61K 9/20; A61K 31/542

(52) U.S. Cl. CPC A61K 9/2013; A61K 31/542; A61K 9/2009
USPC 424/465; 514/226.5; 427/2.14

(57) ABSTRACT

The present invention discloses a fast dissolving pharmaceutical composition comprising lornoxicam or pharmaceutically acceptable salts thereof as an active ingredient along with at least one alkalinizer, at least one organic acid and at least one pharmaceutically acceptable excipient. The present invention also discloses processes of preparing fast dissolving pharmaceutical composition.

### FIELD OF THE INVENTION

The present invention relates to an oral pharmaceutical composition which facilitates the rapid dissolution of the therapeutic compound from the dosage form with subsequent rapid absorption. The present invention also relates to a process for preparing the same.

### BACKGROUND OF THE INVENTION

Lornoxicam (chlortenoxicam) is a potent non-steroidal anti-inflammatory drug (NSAID) of the oxicam class. The chemical name of Lornoxicam is (3E)-6-chloro-3-[hydroxy(pyridin-2-ylamino)methylene]-2-methyl-2,3-dihydro-4H-thieno[2,3-e][1,2]thiazin-4-one 1,1-dioxide. Lomoxicam has the following structure represented by formula I:

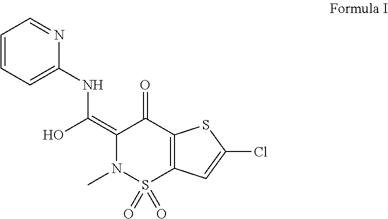 [SMILES: CN1/C(=C(\O)Nc2ccccn2)C(=O)c2sc(Cl)cc2S1(=O)=O]

Lornoxicam has analgesic, anti-inflammatory and antipyretic properties. It is available in oral and parenteral formulations and is used for inflammatory diseases of joints, osteoarthritis, pain following surgery and pain in the lower back and hip which travels down the back of the thigh into the leg (sciatica). Lornoxicam differs from other oxicam compounds in its potent inhibition of prostaglandin biosynthesis through its effects on the enzyme cyclo-oxygenase (COX), a property that explains the particularly pronounced efficacy of the drug. COX-1 is the enzyme associated with gastric mucosal protection while COX-2 is involved in pain and inflammation, and lornoxicam provides balanced inhibition of both isoforms i.e., COX1/COX2. Lornoxicam readily penetrates into synovial fluid producing synovial fluid: plasma AUC ratios in the region of 0.5 after administration of 4 mg twice daily.

In contrast to the other oxicams, lornoxicam has a very short half-life (approximately 4 hours as compared with >24 hours for the others) and is therefore especially suitable for short-term treatment. The short half-life of the drug probably explains the improved gastrointestinal safety profile observed with lornoxicam.

Lomoxicam is slightly lipophilic with an apparent partition coefficient (n-octanol/buffer pH 7.4) of 1.8 and a pKa of 4.7. As a result of keto-enol-tautomerism where the basic pyrimido group with a pKa value of 5.5 increases the acidity of the enolic group to a pKa of 1-2, lornoxicam is amphoteric & exists as a zwitter ion in the physiological pH range 2-5, and as an anion at pH values equal to and above 6.

Lornoxicam has very low solubility under acidic conditions such as found in the stomach, less than 1 mg/100 ml 0.1 N HCl at room temperature (Bertelsen et al U.S. Pat. No. 6,713,089). Experimentally it was found that the solubility of lornoxicam was 0.0056 mg/ml at pH 1.2 and 0.0104 mg/ml in water. This means that in acidic conditions such as found in the stomach, an 8 mg dose of lornoxicam will require more than 1400 ml of acid for complete dissolution. This far exceeds typical volumes present in the stomach when a tablet is swallowed with around 150-250 ml of co-administered water. Such low solubility causes slow and variable absorption of lornoxicam in vivo which takes around 2.5 hours to reach peak plasma concentrations (Tₘₐₓ), ranging from 1.4-6 hours, in fasted subjects.

Accordingly, increasing the rate of dissolution and rate of absorption of lornoxicam should enable greater and more rapid analgesic, anti-inflammatory and antipyretic effects after oral dosing. In this regard, oral delivery is the most convenient and acceptable route of drug administration for patients. A tablet or capsule of a suitable small size for swallowing whole is preferred to one which requires prior dispersion or dissolution in water prior to administration. A solid swallow dosage form avoids taste problems as lornoxicam has an unpleasant bitter taste which is more pronounced when the drug is dissolved.

From a pharmaceutical manufacturing perspective, a fast dissolving and fast acting formulation must be produced using traditional tablet manufacturing techniques such as direct compression or wet granulation without the need for specialized processing steps. Tablets must be physically robust with adequate strength and integrity to withstand the processes of tabletting, film coating such as rotating in a pan and automatic packaging. As well as the tablet must demonstrate good chemical and physical stability.

Improving the rate and extent of absorption of oral formulations of compounds has been the subject of substantial research. In general, once a solid dosage form reaches the stomach, it undergoes disintegration and/or dissolution. The resultant solution then passes into the small intestine where the active ingredient can be absorbed through the intestinal mucosa into the circulatory system. The drug is then carried via the portal vein and liver to the site of action.

The time delay for in vivo disintegration and dissolution can be reduced by dispersion or dissolution of the drug before swallowing. Effervescent tablets or powders are well known examples of such products comprising large amounts of an organic acid such as citric acid with alkaline agents such as sodium bicarbonate, sodium carbonate and/or calcium carbonate. These effervescent formulations can be dispersed and/or dissolved prior to administration or chewed before swallowing as disclosed in the prior art.

For example, U.S. Pat. No. 6,245,353 (Tritthart) discloses dispersible, soluble and chewable effervescent tablets containing cetirizine and an effervescent couple comprising a base and an acid. The compressed tablets exemplified in U.S. Pat. No. 6,245,353 typically contain 32-47% by weight of the base where tablets are from 890-2343 mg. U.S. Pat. No. 4,704,269 (Korab) discloses soluble effervescent tablets weighing 4.5 g containing 40-60% by weight of an antacid with 35-55% by weight of an organic acid. U.S. Pat. No. 4,309,408 (Pathak) discloses effervescent powder mixes containing paracetamol with metoclopramide where the unit dose is 2.3-3.1 g and the powder contains 22-49% by weight of the effervescent base. U.S. Pat. No. 4,942,039 (Duvall) discloses an effervescent tablet weighing 2,976 mg containing 6.25 mg of ketoprofen and 35% by weight of carbonates and bicarbonates. Effervescent products described in the prior art typically contain less than 50% by weight of the base and, with the high loading of effervescent couple the products are too large to be swallowed whole if presented as a compressed tablet.

It is widely accepted that for many drugs, the solubility can be significantly increased by changing the pH and when the solubility is increased, an increase in the dissolution rate occurs. Enhanced absorption of acidic NSAIDs such as aspirin, naproxen, diflunisal and ibuprofen has been reported in the presence of common antacids such as sodium bicarbonate and magnesium hydroxide. These antacids increase the gastric pH and thus increase the solubility of these drugs resulting in faster absorption. While increased solubility and enhanced absorption occurs for acidic drugs with an alkaline agent which increases the pH, this will not occur for all drugs. For basic drugs, solubility decreases with increased pH, so any increase in gastric pH tends to reduce the dissolution rate.

Although for unionized drugs such as paracetamol, the solubility is independent of pH, U.S. Pat. No. 6,316,025 (Grattan) discloses the use of high levels of sodium bicarbonate (300 mg to 1000 mg per tablet) to enhance the absorption of paracetamol. This is attributed to a prokinetic effect of the isotonic solution that results when two tablets each containing 500 mg paracetamol with 630 mg sodium bicarbonate are ingested with 100 ml of water. It is proposed that the enhanced absorption results from the stimulation of gastric emptying by the isotonic solution rather than a pH effect on solubility. U.S. Pat. No. 6,316,025 teaches tablets with a drug to sodium bicarbonate ratio greater than 0.74:1, where tablets contain around 50% bicarbonate. A similar, approach is described for the basic drug eletriptan in US Patent Application No. 20040204475 (Humphrey), using 630 mg sodium bicarbonate per tablet to obtain a duodenal concentration approximately isotonic with serum (150 millimoles).

In relation to acidic drugs, particularly acidic NSAIDs and their salts, of which lornoxicam is an example, the prior art deals with the use of sodium bicarbonate and other pH modulating agents at both low and high levels of alkaline agents where the effect on solubility will be quite different.

For diclofenac, WO 97/44023 and U.S. Pat. No. 6,974,595 (Reiner) teach the use of sodium and potassium bicarbonates at a level of 20-80% by weight of the drug which equates to 10-mg bicarbonate for a 50 mg diclofenac potassium tablet. Soluble and solid dosage forms are exemplified and pharmacokinetic data demonstrate that absorption from formulations with low levels of bicarbonate produced was more consistent and produced less variable plasma levels compared with tablets without bicarbonate. Shorter mean Tₘₐₓ values of 21.2 and 29.8 minutes respectively were obtained for 25 mg and 50 mg film-coated tablets with bicarbonate compared with a mean Tₘₐₓ of 50.8 minutes for the standard immediate release tablets.

Specifically for lornoxicam, prior art teaches the use of similarly low levels of bicarbonate (32-40 mg) with 8 mg lornoxicam to improve the in vitro dissolution rate compared with the standard tablets. In both cases, special processing of the drug with the alkaline agent is required and there is an explicit teaching that the observed fast dissolution of the drug cannot be achieved using traditional direct compression or wet granulation processing techniques.

U.S. Pat. No. 6,713,089 (Bertelsen) discloses the reaction of the drug and alkali in the presence of water to increase the in vitro dissolution to at least 50% in 20 minutes in 1300 ml 0.1 N HCl at 50 rpm. The reaction in the presence of water is stated to be essential to achieve the claimed dissolution which is not achieved in traditional tablet formulations. As an improvement on the aqueous treatment, US Patent Application No. 20070218128 (Bertelsen) describes the co-milling or equivalent intimate mixing of drug and alkaline substances either without or with a minimum amount of liquid to achieve fast in vitro dissolution of at least 50% within the first 20 minutes. Such mixtures prepared without liquids produced tablets, which had water contents similar to batches of tablets prepared by wet granulation, and yet showed improved stability despite the comparable water content. Again it is stated that the intimate mixing of drug and alkaline agent is essential for fast dissolution which is not achieved with traditional tablet processing. These new formulations which contain 40% bicarbonate were shown to reduce the median Tₘₐₓ for lornoxicam from 2.5 hours from standard tablets, to 0.5 hours demonstrating faster absorption when the lornoxicam was co-processed intimately with the bicarbonate.

Although lornoxicam is not exemplified, fast dissolving swallow tablets of acidic drugs containing high levels of bicarbonate are disclosed in the prior art. PCT/AU2006/001798 (Imaginot) discloses the use of up to 10 mmoles of a soluble carbonate with a range of acidic drugs including three NSAIDs. Ibuprofen, naproxen and diclofenac are the free acids which have a lower aqueous solubility than the more soluble sodium or potassium salts. These free acids are comparable with the drug of the present invention which is the low solubility free acid lornoxicam. Examples of these NSAID free acids show that when formulated with up to 600 mg sodium bicarbonate without any additional acid, more than 66% dissolution of the drug can be achieved in 3 minutes in 900 ml 0.0033 M HCl at 30 rpm in USP dissolution apparatus II at 37° C.

The prior art suggests that a high level of soluble carbonate can be used to maximize the dissolution rate for lornoxicam. However, subsequent investigations have shown that lornoxicam appears not to achieve those dissolution characteristics with alkalinizer alone.

As sodium bicarbonate per se has relatively poor compression properties, formulations containing a high proportion of bicarbonate will require high levels of fillers and binders in order to produce suitable tablets that are physically robust, sufficient to withstand the commercial processes of tabletting, film coating such as rotating in a pan, and automatic packaging. In turn this effectively increases the size of the tablet making it more difficult to swallow.

In addition, higher compaction forces to produce a less friable and harder tablet will tend to increase the disintegration and dissolution times of the tablet which can delay drug absorption. US Patent Application 20020034540 (Price et al) discloses improved physical properties of tablets containing at least 35% by weight ibuprofen with 25-75 mg alkali metal carbonate or bicarbonate where the crushing strength of the tablet is 6.5-15 Kp. The low levels of carbonate or bicarbonate reduces the disintegration time to less than 10 minutes.

In spite of the existing prior art mentioned above, there is still need for an invention that provides a small, easy-to-swallow oral formulation which facilitates the rapid delivery of the therapeutic compound to the circulatory system for fast onset of action. Such a formulation must be capable of manufacture by conventional pharmaceutical processes without the need for specialized or expensive equipment or processing. Furthermore, it is important for such fast acting swallow formulations to consistently achieve fast dissolution and absorption even in the absence of gastric acid, under the wide range of different conditions that occur in the gastrointestinal tract in the general population, ranging from pH 1-7. It is also desirable that such formulations will be effective when administered with limited volumes of liquid and when there is gut stasis which is often associated with pain and migraine and tends to slow in vivo dissolution and absorption.

### OBJECTS OF THE INVENTION

It is an object of the present invention to provide an oral pharmaceutical composition which facilitates the rapid dissolution of the therapeutic compound from the dosage form with subsequent rapid absorption.

It is another object of the present invention to provide a fast dissolving composition containing NSAID having low solubility such as lornoxicam.

It is still another object to provide a fast dissolving composition containing a combination of pH modulating agents in a specific proportion.

It is yet another object of the present invention to provide a fast dissolving composition in the form of tablet in which the amount of pharmaceutically acceptable excipients such as fillers or diluents and binders is effectively restricted to obtain relatively small easy to swallow tablets.

### SUMMARY OF THE INVENTION

In accordance with the present invention there is provided a fast dissolving pharmaceutical composition comprising:
a. lornoxicam or pharmaceutically acceptable salts thereof;
b. at least one alkalinizer;
c. at least one organic acid; and
d. pharmaceutically acceptable excipients
wherein the weight ratio of lornoxicam to alkalinizer(s) is in the range of from about 1:43 to about 1:85 and the weight ratio of alkalinizer(s) to organic acid(s) is in the range of from about 3:1 to about 100:1;
wherein at least 30% of lornoxicam is released from said composition within 3 minutes and at least 40% of lornoxicam is released from said composition within 10 minutes in a USP type II apparatus at 30 rpm using 0.0033 M HCl as a dissolution medium at 37±0.5° C.

Typically, the amount of lornoxicam or pharmaceutically acceptable salts thereof is in the range of about 0.5% to about 2.5% of the mass of the composition.

Typically, the alkalinizer is at least one selected from the group comprising sodium bicarbonate, potassium bicarbonate, calcium carbonate, sodium carbonate and combinations thereof.

In accordance with one of the embodiments of the present invention the alkalinizer used is a bicarbonate.

In accordance with another embodiment of the present invention the alkalinizer used is a mixture of bicarbonate and carbonate.

Typically, the amount of alkalinizer is in the range of about 43% to about 85% of the mass of the composition.

Typically, the organic acid is at least one selected from the group comprising fumaric acid, citric acid, tartaric acid, succinic acid, glycine and combinations thereof.

Typically, the amount of the organic acid is in the range of about 3% to about 20% of the mass of the composition.

Typically, the composition of the present invention exhibits a lornoxicam plasma Tₘₐₓ of less than 45 minutes. (Tₘₐₓ: The mean time to maximum blood plasma concentration)

Typically, the composition of the present invention exhibits a lornoxicam plasma Cₘₐₓ of more than 950 ng/ml after the administration of a 8 mg dose of lornoxicam. (Cₘₐₓ: Peak plasma concentration)

Typically, the composition of the present invention exhibits a lornoxicam AUC_{(0-10 min)} of about 10 ng.h/ml to about 70 ng.h/ml after the administration of a 8 mg dose of lornoxicam.

Typically, the composition of the present invention exhibits a lornoxicam AUC_{(0-20 min)} of about 70 ng.h/ml to about 200 ng.h/ml. after the administration of a 8 mg dose of lornoxicam.

Typically, the composition of the present invention exhibits a lornoxicam AUC_{(0-30 min)} of about 200 ng.h/ml to about 500 ng.h/ml after the administration of a 8 mg dose of lornoxicam.

In accordance with another embodiment of the present invention the composition further comprises at least one water uptake agent selected from the group comprising cross-linked polyvinylpyrrolidone, croscarmellose sodium, sodium starch glycolate, starch, starch derivatives, hydroxypropylcellulose, low substituted hydroxypropylcellulose, hydroxypropylmethyl cellulose, alginic acid, sodium alginate, calcium sulphate, calcium carboxymethylcellulose, microcrystalline cellulose, powdered cellulose, colloidal silicon dioxide, docusate sodium, guar gum, magnesium aluminium silicate, methylcellulose, polarcrilin potassium, silicified microcystalline cellulose, magnesium oxide, tragacanth, mannitol, sorbitol, xylitol, sucrose, lactose, fructose, maltose, polyethylene glycol, amino acids, cyclodextrin, urea, polyvinylpyrrolidone and combinations thereof.

Typically, the amount of water uptake agent is in the range of about 20% to about 50% of the mass of the composition.

In accordance with the preferred embodiment of the present invention there is provided a fast dissolving pharmaceutical composition comprising:
a) lornoxicam or pharmaceutically acceptable salts thereof in an amount of about 0.5% to about 2.5% of the mass of the composition;
b) at least one alkalinizer;
c) at least one organic acid; and
d) pharmaceutically acceptable excipients,
wherein the weight ratio of lornoxicam to alkalinizer(s) is in the range of from about 1:43 to about 1:85 and the weight ratio of alkalinizer(s) to organic acid(s) is in the range of from about 3:1 to about 100:1,
wherein said composition exhibits a dissolution profile within the following ranges:

| Time | lornoxicam |
|---|---|
| 3 minutes | at least 30% |
| 10 minutes | at least 40% |

in a USP type II apparatus at 30 rpm using 0.0033 M HCl as a dissolution medium at 37±0.5° C.

Typically, the pharmaceutically acceptable excipient is at least one selected from the group consisting of disintegrants, preservatives, colors, anti-oxidants, sweeteners, flavoring agents, emulsifiers, binders, glidants and lubricants.

Typically, the composition is formulated in a dosage form selected from the group consisting of tablet, capsule, granules and powder.

In accordance with another aspect of the present invention there is provided a process for the preparation of a fast dissolving pharmaceutical composition; said process comprising the following steps:
(a) mixing lornoxicam or pharmaceutically acceptable salts thereof with at least one alkalinizer and other pharmaceutically acceptable excipients to obtain a mixture;
(b) optionally granulating the mixture;
(c) adding at least one organic acid to the mixture to obtain a blend,
(c) compressing the blend to form a tablet; and
(d) optionally coating the tablet to convert it into pharmaceutically acceptable film coated tablet,
   wherein the weight ratio of lornoxicam:alkalinizer(s) is in the range of from about 1:43 to about 1:85 and the weight ratio of alkalinizer(s):organic acid(s) is in the range of from about 3:1 to about 100:1.

### BRIEF DESCRIPTION OF THE DRAWINGS AND FIGURES

FIG. 1 is a graph illustrating comparative dissolution profile of the present composition and a reference composition available in the market; and

FIG. 2 is a graph illustrating comparative plasma profile of the present composition (T) and a reference composition (R) available in the market.

### DETAILED DESCRIPTION OF THE INVENTION

NSAIDs, for which fast absorption and fast onset of action are required for effective rapid pain relief, typically have low solubility in water and acidic gastric fluid. Low solubility causes slow dissolution which in turn affects the absorption. The limited volume of fluid in the gastrointestinal tract further reduce the extent of dissolution of such drugs and potentially limit the rate at which they will be absorbed. The present invention demonstrates that the dissolution rate of lornoxicam or pharmaceutically acceptable salts thereof can be increased by preparing a relatively small easy-to-swallow tablet.

The present invention provides an oral drug delivery system containing low solubility NSAID drug such as lornoxicam or pharmaceutically acceptable salts thereof. The present invention particularly, focus on a fast dissolving pharmaceutical composition containing lornoxicam or pharmaceutically acceptable salts thereof, in which the solubility of the lornoxicam is enhanced by using an appropriate amount of one or more soluble and/or dispersible pH modulating agents such as alkalinizer(s) and organic acid(s).

In accordance with the present invention there is provided a fast dissolving pharmaceutical composition comprising:
a. lornoxicam or pharmaceutically acceptable salts thereof in an amount of about 0.5% to about 2.5% of the mass of the composition;
b. at least one alkalinizer in an amount of about 43% to about 85% of the mass of the composition;
c. at least one organic acid in an amount of about 3% to about 20% of the mass of the composition; and
d. pharmaceutically acceptable excipients

Typically, the alkalinizer is at least one selected from the group comprising sodium bicarbonate, potassium bicarbonate, calcium carbonate, sodium carbonate and combinations thereof.

In accordance with one of the embodiments of the present invention the alkalinizer used is a bicarbonate.

In accordance with another embodiment of the present invention the alkalinizer used is a mixture of bicarbonate and carbonate.

Typically, the organic acid is at least one selected from the group comprising fumaric acid, citric acid, tartaric acid, succinic acid, glycine and combinations thereof.

In accordance with the present invention the increased level of alkalinizer(s) such as bicarbonates resulted in increased pH and solubility. The maximum % dissolution obtained was around 80% in 900 ml 0.0033 M HCl, when the stirring was increased to 200 rpm for 5 minutes. Under test conditions at 30 rpm, although the dissolution increased to 20% at 5 minutes with 300 mg bicarbonate per tablet and to 30% with 400 mg, further increases in bicarbonate to 500 mg and 600 mg did not produce any further increase in early dissolution despite the higher pH.

It is found that when organic acid(s) such as fumaric acid or citric acid was added, it significantly increases the rate and extent of in vitro dissolution.

It is surprisingly found that through the use of low levels of organic acids such as citric or fumaric acid, the total amount of alkalinizers could be reduced while still achieving significantly increased dissolution rates even when the final pH of the 900 ml 0.0033 M HCl dissolution medium was below pH 6.

In accordance with the preferred embodiment of the present invention the weight ratio of lornoxicam to alkalinizer(s) is in the range of from about 1:43 to about 1:85 and the weight ratio of alkalinizer(s) to organic acid(s) is in the range of from about 3:1 to about 100:1;

In accordance with one of the preferred embodiments of the present invention at least 30% of lornoxicam is released from the present composition within 3 minutes and at least 40% of lornoxicam is released from the present composition within 10 minutes.

In accordance with one of the embodiments of the present invention the composition exhibits a lornoxicam plasma Tₘₐₓ of less than 45 minutes.

The composition of the present invention exhibits a lornoxicam plasma Cₘₐₓ of more than 950 ng/ml after the administration of a 8 mg dose of lornoxicam.

Typically, the composition of the present invention exhibits a lornoxicam AUC_{(0-10 min)} of about 10 ng.h/ml to about 70 ng.h/ml after the administration of a 8 mg dose of lornoxicam.

Typically, the composition of the present invention exhibits a lornoxicam AUC_{(0-20 min)} of about 70 ng.h/ml to about 200 ng.h/ml after the administration of a 8 mg dose of lornoxicam.

Typically, the composition of the present invention exhibits a lornoxicam AUC_{(0-30 min)} of about 200 ng.h/ml to about 500 ng.h/ml after the administration of a 8 mg dose of lornoxicam.

In accordance with another embodiment of the present invention the composition further comprises at least one water uptake agent which facilitate the uptake of water which is selected from the group comprising cross-linked polyvinylpyrrolidone, croscarmellose sodium, sodium starch glycolate, starch, starch derivatives, hydroxypropylcellulose, low substituted hydroxypropylcellulose, hydroxypropylmethyl cellulose, alginic acid, sodium alginate, calcium sulphate, calcium carboxymethylcellulose, microcrystalline cellulose, powdered cellulose, colloidal silicon dioxide, docusate sodium, guar gum, magnesium aluminium silicate, methylcellulose, polarcrilin potassium, silicified microcystalline cellulose, magnesium oxide, tragacanth, mannitol, sorbitol, xylitol, sucrose, lactose, fructose, maltose, polyethylene glycol, amino acids, cyclodextrin, urea, polyvinylpyrrolidone and combinations thereof.

Typically, the amount of water uptake agent is in the range of about 20% to about 50% of the mass of the composition.

The pharmaceutically acceptable excipient is at least one selected from the group consisting of disintegrants, preservatives, colors, anti-oxidants, sweeteners, flavoring agents, emulsifiers, binders, glidants and lubricants.

Typically, the composition is formulated in a dosage form selected from the group consisting of tablet, capsule, granules and powder.

In accordance with another aspect of the present invention there is provided a process for the preparation of a fast dissolving pharmaceutical composition. The process of the present invention is described herein below:

In the first step, lornoxicam or pharmaceutically acceptable salts thereof is mixed with at least one alkalinizer and pharmaceutically acceptable excipients to obtain a mixture. The obtained mixture is used as such for the next step or it is granulated first and then used.

In the next step, at least one organic acid is added to the mixture to obtain a blend.

The obtained blend is then compressed to form a tablet. Optionally the obtained tablet is coated with coating polymer to convert it into pharmaceutically acceptable film coated tablet.

Typically, the weight ratio of lornoxicam:alkalinizer(s) is in the range of from about 1:43 to about 1:85 and the weight ratio of alkalinizer(s):organic acid(s) is in the range of from about 3:1 to about 100:1.

The fast dissolving pharmaceutical compositions of the present invention for lornoxicam or pharmaceutically acceptable salts thereof can be prepared by techniques like wet granulation or direct compression. In wet granulation, lornoxicam is mixed with at least one alkalinizer(s) like sodium bicarbonate or sodium carbonate and suitable pharmaceutically acceptable excipients followed by granulation using suitable solvent and addition of organic acid. Direct compression involves physical admixture of lornoxicam with at least one alkalinizer(s) like sodium bicarbonate or sodium carbonate and at least one organic acid(s) like fumaric acid or citric acid. Said granulates or physical admixture is compressed using appropriate tablet tooling or filled in suitable capsule or sachet to obtain desired pharmaceutical dosage form of the present invention.

### DEFINITIONS

The term “pharmaceutical composition” as used herein is pharmaceutical formulations or pharmaceutical dosage forms comprising lornoxicam or pharmaceutically acceptable salts thereof.

The term “stable” as used herein refers to the physicochemical stability of lornoxicam when kept at 40° C. & 2° C., 75% RH±5% RH for 3 months, when there is no change in the physical appearance of the pharmaceutical composition and complies assay and drug release profiles.

The term “excipients” as used herein means a component of a pharmaceutical product that is not an active ingredient for example, disintegrants, fillers, diluents, carriers, alkalinizers, plasticizers, antiadherents, glidants, binders, solvents and the like. The excipients that are useful in preparing a pharmaceutical composition are safe, non-toxic and are acceptable for pharmaceutical use.

The term “alkalinizer” as used herein means inert substances used as a pH modulator to increase the pH. Such compounds include, by way of example and without limitation, sodium bicarbonate, potassium bicarbonate, calcium carbonate, sodium carbonate, combinations thereof and other such materials known to those skilled in the art.

The term “organic acid” as used herein means inert substances used as a pH modulator to decrease the pH. Such compounds include, by way of example and without limitation, fumaric acid, citric acid, tartaric acid, succinic acid, glycine, combinations thereof and other such materials known to those skilled in the art.

The term “diluent” or “filler” as used herein means inert substances used as fillers to create the desired bulk, flow properties. Such compounds include, by way of example and without limitation, microcrystalline cellulose, dibasic calcium phosphate, mannitol, pregelatinized starch, sucrose, powdered cellulose, precipitated calcium carbonate, starch, lactose, glucose, calcium carbonate, calcium sulphate, magnesium carbonate, combinations thereof and other such materials known to those skilled in the art.

The term “binder” as used herein means agents used in making granules of the active ingredient by mixing it with diluents/fillers. Such compounds include, by way of example and without limitation, polyvinylpyrrolidone (PVP, povidone), hydroxypropyl cellulose (HPC), pregelatinized starch, starch, hydroxylpropyl methyl cellulose (HPMC), and hydroxy ethyl cellulose (HEC), combinations thereof and other such materials known to those skilled in the art.

The term “lubricant” as used herein means agents used in formulations to improve flow-properties. Compounds include, by way of example and without limitation, magnesium stearate, stearic acid, calcium stearate, sodium stearyl fumarate, sodium lauryl sulphate, magnesium lauryl sulphate, sodium benzoate, hydrogenated vegetable oil, combinations thereof and other such materials known to those skilled in the art.

The term “disintegrant” as used herein means agents used in formulations to improve disintegration and hence dissolution. Such compounds include, by way of example and without limitation, starch and starch derivatives, sodium starch glycolate, alginic acid, sodium alginate, crospovidone, croscarmellose sodium, insoluble ion exchange resins, combinations thereof and other such materials known to those skilled in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

The following examples are provided to enable one skilled in the art to practice the invention and are merely illustrative of the present invention. The examples should not be read as limiting the scope of the present invention.

### EXAMPLES

### Example-1

The present fast dissolving pharmaceutical composition for lornoxicam or pharmaceutically acceptable salts thereof can be prepared as shown in table 1 and described below.

TABLE 1

| Sr. No. | Ingredients | mg/tablet | % w/w |
|---|---|---|---|
| 1 | Lornoxicam | 8.0 | 1.0 |
| 2 | Microcrystalline cellulose (Avicel PH 102) | 118.0 | 14.7 |
| 3 | Sodium bicarbonate | 500.0 | 62.5 |
| 4 | Sodium starch glycolate | 40.0 | 5.0 |
| 5 | Povidone K30 | 24.0 | 3.0 |
| 6 | Isopropyl alcohol | q.s | q.s |
| 7 | Fumaric acid | 50.0 | 6.3 |
| 8 | Sodium starch glycolate | 56.0 | 7.0 |
| 9 | Magnesium stearate | 4.0 | 0.5 |
| | Total weight | 800.0 | 100.0 |
| | Film coating | | |
| 10 | Opadry ® II yellow 85F520005 | 3.0 | 24.0 |
| 11 | Purified water | q.s | q.s |
| | Total weight | | 824.0 |

### Procedure

1. Accurately weighed lornoxicam, microcrystalline cellulose (Avicel PH 102), sodium bicarbonate and sodium starch glycolate were passed through sieve (#40) and mixed well to obtain a blend.
2. The obtained blend was granulated in a rapid mixer granulator using Povidone K30 solution (30% w/v) in isopropyl alcohol as binder to obtain wet granules.
3. The wet granules were kept for drying in a tray drier at 45°-50° C. to obtain dried granules.
4. The dried granules were sifted through #40 sieve.
5. These granules were blended with #40 sieve passed sodium starch glycolate and fumaric acid.
6. The obtained blend was lubricated with #60 sieve passed magnesium stearate to obtain a final blend.
7. The final blend was compressed by using 16.6×7.6 mm capsule shaped punch, plain on both side to obtain tablets.
8. The obtained tablets were film coated using Opadry® II yellow 85F520005 (20% w/v) in purified water.

Tablet parameters-

| | |
|---|---|
| Tablet weight- | 824 mg |
| Hardness- | 13.0 kg/cm² |
| Thickness- | 5.25 mm |
| Disintegration time- | 165 seconds |

### Example-2

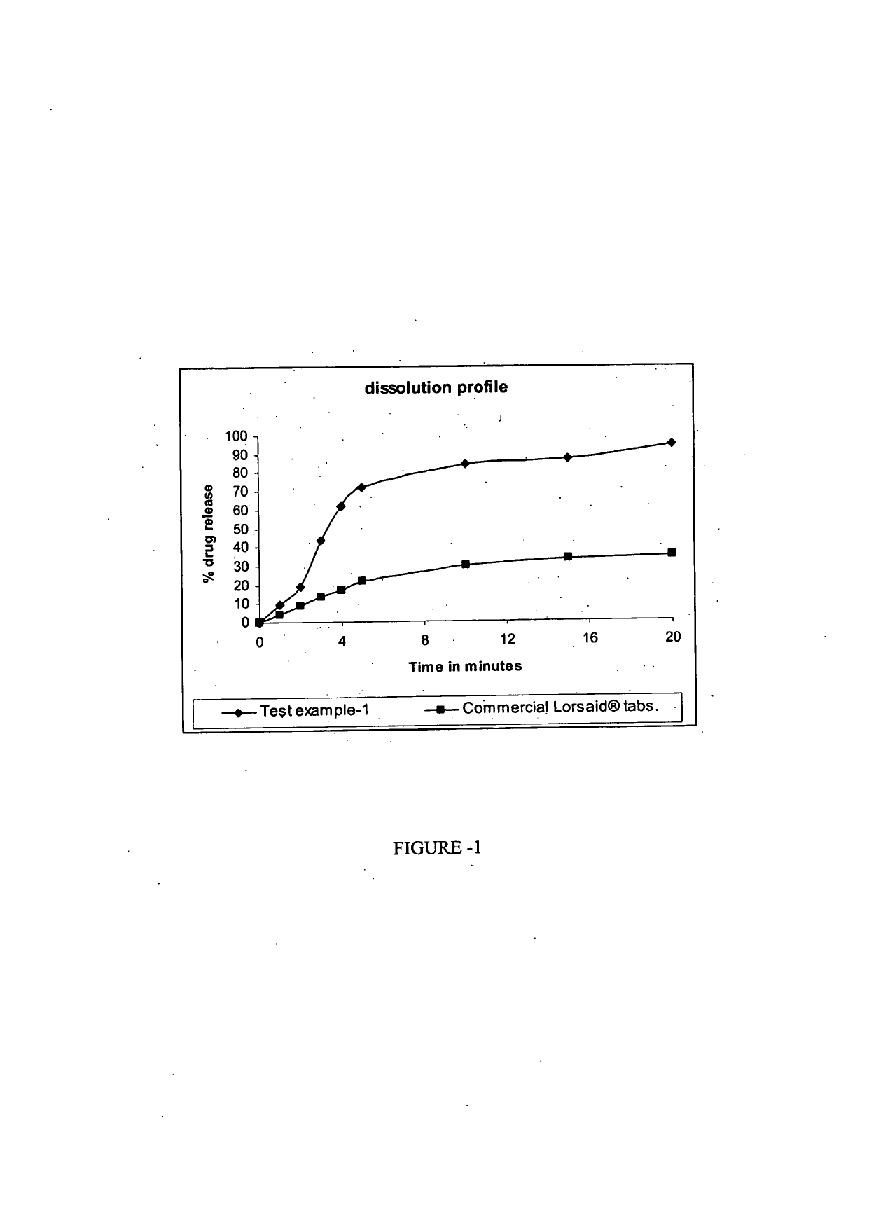

The pharmaceutical composition as prepared in example-1 was compared with commercial tablets for dissolution and the results obtained are shown in table 2 and fig 1.

### Dissolution Conditions

USP dissolution apparatus II (Paddle), 900 ml, 0.0033M HCl, 37° C.±0.5° C., rpm for 0-15 minutes followed by 200 rpm till 20 minutes.

TABLE 2

% dissolution (% release)

| Time in | | Commercial |
|---|---|---|
| minutes | Test example -1 | Lorsaid ® tabs. |
| 0 | 0.0 | 0.0 |
| 1 | 9.71 | 3.9 |
| 2 | 19.38 | 8.9 |
| 3 | 43.58 | 13.4 |
| 4 | 61.98 | 17.3 |
| 5 | 72.24 | 21.9 |
| 10 | 84.64 | 30.0 |
| 15 | 86.95 | 33.3 |
| 20 | 94.80 | 35.0 |

### Example-3

### Pharmacokinetic Studies

The pharmaceutical composition as prepared in example-1 (herein after defined as test composition) was compared with a lornoxicam composition i.e. LORSAID® (herein after defined as reference composition) which is already available in the market. 24 healthy volunteers were randomized to receive 8 mg of the two products (either test composition or reference composition). Each drug administration was separated by a washout period of seven days.

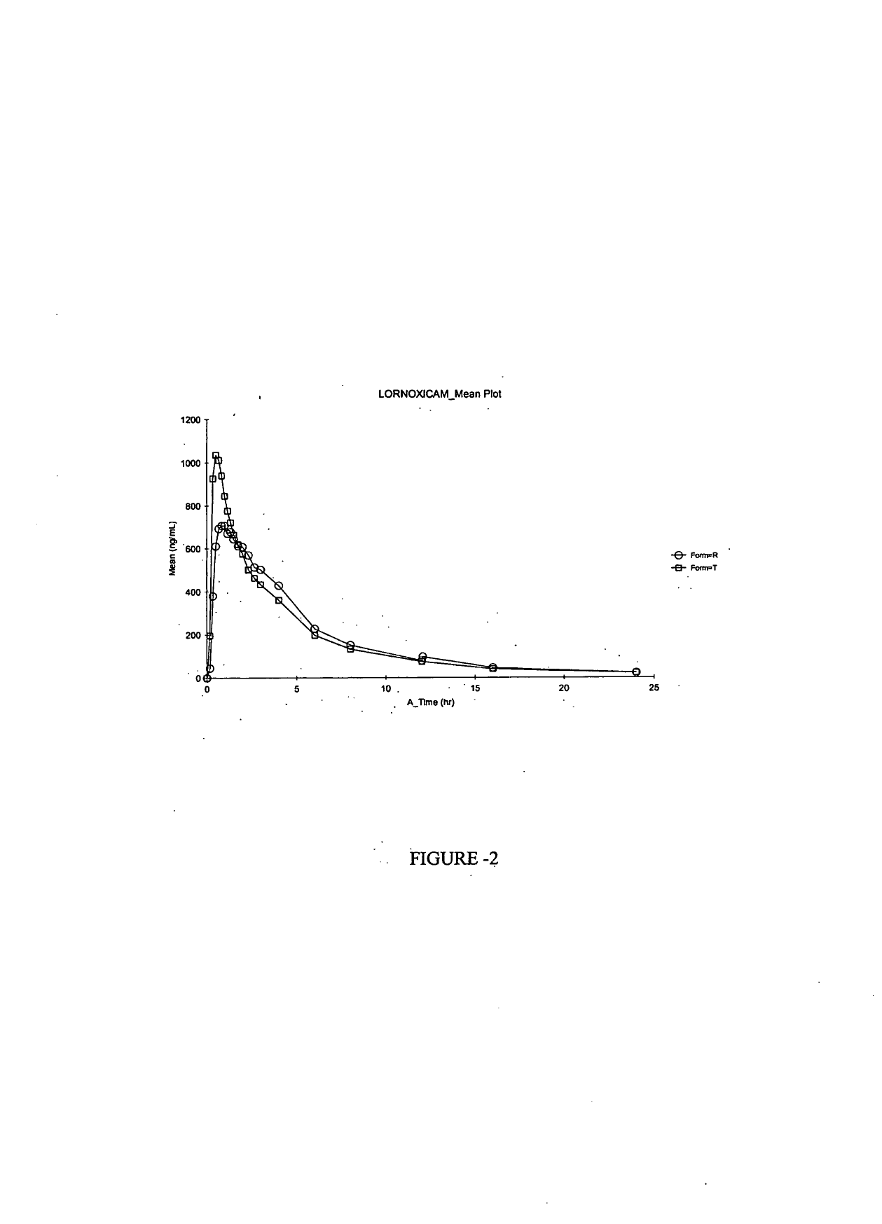

Blood samples (5 ml) were obtained from subjects at 0 (pre dose), at 10, 20, 30, 40, 50, 60, 70, 80, 90, 105, 120, 140, 160 minutes, 3.00, 4.00, 6.00, 8.00, 12.00, 16.00 and 24.00 hours post-dose in each period. Plasma concentrations of lornoxicam were determined using a validated LC-MS/MS method. Mean plasma concentration time profiles are shown in FIG. 2 and mean values of pharmacokinetic parameters of lornoxicam obtained from this study are presented in table 3.

TABLE 3

| | Composition | | | | | |
|---|---|---|---|---|---|---|
| | Cₘₐₓ | Tₘₐₓ | AUC₍₀₋₁₀₎ | AUC₍₀₋₂₀₎ | AUC₍₀₋₃₀₎ | AUC_{(0-∞)} |
| | (ng/ml) | (hours) | (ng · h/ml) | (ng · h/ml) | (ng · h/ml) | (ng · h/ml) |
| Test | 1097.78 | 0.514 | 17.03 | 106.89 | 273.61 | 4182.58 |
| Reference | 787.82 | 1.056 | 4.20 | 38.42 | 122.23 | 4257.26 |
| Ratio: Test/ | 1.39 | 0.49 | 4.06 | 2.78 | 2.24 | 0.98 |
| Reference | | | | | | |

### Example-4

### Stability Studies

The pharmaceutical composition as prepared in example-1 was kept for stability studies at 40° C.±2° C., 75% RH±5% RH for 3 months and the results are shown in table 4.

TABLE 4

| Test | Specification | Condition | | | |
|---|---|---|---|---|---|
| | | Initial | 40° C. ± 2° C., | | |
| | | | 75% RH ± 5% RH | | |
| | | | 1M | 2M | 3M |
| Description | Yellow colored, | Com- | Com- | Com- | Com- |
| | oblong shaped, coated | plies | plies | plies | plies |
| | tablets, plain on both | | | | |
| | sides | | | | |
| Assay | Not less than 90.0% | 97.58 | 93.20 | 94.56 | 93.23 |
| | and not more than | | | | |
| | 110.0% | | | | |

Dissolution - USP type II apparatus (Paddle), 900 ml, 0.0033M HCl,
37 ± 0.5° C., 30 rpm from 0-15 minutes followed by 200 rpm to 20
minutes.

| Time (Min) | | % dissolution (% release) | | | |
|---|---|---|---|---|---|
| 1 | | 9.7 | 1.0 | 1.0 | 2.0 |
| 2 | | 19.3 | 11.0 | 18.0 | 14.0 |
| 3 | | 43.5 | 31.0 | 35.0 | 36.0 |
| 4 | | 61.9 | 50.0 | 58.0 | 57.0 |
| 5 | not less than 50% | 72.2 | 68.0 | 70.0 | 71.0 |
| 10 | | 84.6 | 84.0 | 87.0 | 82.0 |
| 15 | not less than 75% | 86.9 | 87.0 | 87.0 | 85.0 |
| 20 | | 94.8 | 98.0 | 92.0 | 94.0 |

### Example-5

The effect of different alkalinizers on lornoxicam dissolution when used in combination with fumaric acid was studied. Table 5 shows different compositions and the results obtained are shown in table 6.

TABLE 5

| Sr. | | | | | | |
|---|---|---|---|---|---|---|
| No. | Ingredients (%) | 1584/059 II | 1584/103 | 1584/105 | 1584/111 | 1584/121 |
| 1 | Lornoxicam | 1.0 | 1.0 | 1.0 | 1.0 | 1.0 |
| 2 | Sodium | 62.5 (500 mg) | 31.3 (250 mg) | — | — | 31.3 (250 mg) |
| | Bicarbonate | | | | | |
| 3 | Calcium | — | 31.3 (250 mg) | 62.5 (500 mg) | — | — |
| | carbonate | | | | | |
| 4 | Sodium | — | — | — | 62.5 (500 mg) | 31.3 (250 mg) |
| | Carbonate | | | | | |
| 5 | Fumaric acid | 6.2 (50 mg) | 6.2 (50 mg) | 6.2 (50 mg) | 6.2 (50 mg) | 6.2 (50 mg) |
| 6 | MCC (Avicel | 24.8 | 24.8 | 24.8 | 24.8 | 24.8 |
| | PH200) | | | | | |
| 7 | Sodium starch | 5.0 | 5.0 | 5.0 | 5.0 | 5.0 |
| | glycolate | | | | | |
| 8 | Magnesium | 0.5 | 0.5 | 0.5 | 0.5 | 0.5 |
| | stearate | | | | | |
| | TOTAL | 100.0 | 100.0 | 100.0 | 100.0 | 100.0 |

TABLE 6

| | % dissolution (% release) | | | | |
|---|---|---|---|---|---|
| Time | 1584/059 II | 1584/103 | 1584/105 | 1584/111 | 1584/121 |
| in Min. | SBC | SBC + CC | CC | SC | SBC + SC |
| 0 | 0.0 | 0.0 | 0.0 | 0.0 | 0.0 |
| 1 | 44.2 | 33.6 | 14.7 | 2.1 | 10.6 |
| 2 | 52.1 | 46.0 | 17.8 | 4.2 | 18.4 |
| 3 | 57.0 | 53.3 | 20.9 | 5.9 | 25.6 |
| 4 | 61.1 | 57.8 | 23.7 | 6.3 | 30.4 |
| 5 | 60.0 | 60.8 | 26.6 | 7.1 | 33.7 |
| 10 | 72.2 | 72.1 | 37.6 | 11.7 | 41.9 |
| 15 | 76.7 | 69.8 | 44.0 | 13.6 | 49.9 |
| 20 | 87.9 | 78.6 | 72.7 | 72.5 | 96.8 |
| pH | 6.25 | 6.25 | 6.30 | 8.76 | 6.85 |

The results as shown in table 6 clearly indicate that the alkalinizers such as bicarbonate alone, calcium carbonate alone and combination of carbonate & bicarbonate are effective/useful for the purpose of the present invention.

### Example-6

The effects of various acids (6.25-6.3% of the mass of the composition) such as citric acid, fumaric acid, tartaric acid, succinic acid or glycine when used in combination with sodium bicarbonate on lornoxicam dissolution were successfully studied. The different compositions are shown in table No. 7 and the results obtained are shown in table 8.

TABLE 7

| Sr. | | | | | | |
|---|---|---|---|---|---|---|
| No. | Ingredients (%) | I | II | III | IV | V |
| 1 | Lornoxicam | 1.0 | 1.0 | 1.0 | 1.0 | 1.0 |
| 2 | Sodium | 62.50 (500 mg) | 62.50 (500 mg) | 62.50 (500 mg) | 62.50 (500 mg) | 62.50 (500 mg) |
| | Bicarbonate | | | | | |
| 3 | Citric acid | 6.25 (50 mg) | — | — | — | — |
| | anhydrous | | | | | |
| 4 | Fumaric acid | — | 6.3 (50 mg) | — | — | — |
| 5 | Tartaric acid | — | — | 6.3 (50 mg) | — | — |
| 6 | Succinic acid | — | — | — | 6.3 (50 mg) | — |
| 7 | Glycine | — | — | — | — | 6.25 (50 mg) |
| 8 | MCC (Avicel | 24.75 | 24.8 | 24.8 | 24.8 | 24.75 |
| | PH102) | | | | | |
| 9 | Sodium starch | 5.0 | 5.0 | 5.0 | 5.0 | 5.0 |
| | glycolate | | | | | |
| 10 | Magnesium | 0.5 | 0.5 | 0.5 | 0.5 | 0.5 |
| | stearate | | | | | |
| | TOTAL | 100.0 | 100.1 | 100.1 | 100.1 | 100.0 |

TABLE 8

| | % dissolution (% release) | | | | |
|---|---|---|---|---|---|
| | I | II | III | IV | |
| Time | Citric acid | Fumaric | Tartaric | Succinic | V |
| in Min. | anhydrous | acid | acid | acid | Glycine |
| 0 | 0.0 | 0.0 | 0.0 | 0.0 | 0.0 |
| 1 | 14 | 44.2 | 35.1 | 22.4 | 21 |
| 2 | 44 | 52.1 | 51.3 | 41.1 | 46 |
| 3 | 49 | 57.0 | 58.1 | 42.7 | 52 |
| 4 | 53 | 61.1 | 59.4 | 47.1 | 53 |
| 5 | 52 | 60.0 | 59.2 | 50.8 | 55 |
| 10 | 62 | 72.2 | 72.2 | 61.0 | 63 |
| 15 | 70 | 76.7 | 76.3 | 64.2 | 67 |
| 20 | 93 | 87.9 | 80.4 | 71.9 | 88 |

### Example-7

The effect of Fumaric acid (3.1-12.5% of the mass of the composition) when used in combination with sodium bicarbonate on lornoxicam dissolution was studied and the results obtained are shown in table 9.

TABLE 9

| Sr. | | | | | Time in | % dissolution (% release) | | |
|---|---|---|---|---|---|---|---|---|
| No. | Ingredients (%) | 1584/019 II | 1584/055 II | 1584/049 II | Min. | 1584/019 II | 1584/055 II | 1584/049 II |
| 1 | Lornoxicam | 1.0 | 1.0 | 1.0 | 0 | 0.0 | 0.0 | 0.0 |
| 2 | Sodium | 62.5 (500 mg) | 62.5 (500 mg) | 62.5 (500 mg) | 1 | 12.7 | 26.2 | 17.4 |
| | Bicarbonate | | | | | | | |
| 3 | Furmaric acid | — | 3.1 (25 mg) | 12.5 (100 mg) | 2 | 18.3 | 38.5 | 35.4 |
| 4 | MCC (Avicel | 31.0 | 27.9 | 18.5 | 3 | 23.1 | 39.1 | 49.5 |
| | PH200) | | | | | | | |
| 5 | sodium starch | 5.0 | 5.0 | 5.0 | 4 | 25.5 | 44.4 | 55.9 |
| | glycolate | | | | | | | |
| 6 | Magnesium | 0.5 | 0.5 | 0.5 | 5 | 28.8 | 52.2 | 60.1 |
| | stearate | | | | | | | |
| 7 | TOTAL | 100.0 | 100.0 | 100.0 | 10 | 39.0 | 62.1 | 64.8 |
| 8 | — | — | — | — | 15 | 47.8 | 66.4 | 71.4 |
| 9 | — | — | — | — | 20 | 80.3 | 83.3 | 87.5 |
| 10 | — | — | — | — | pH | 6.20 | 6.25 | 6.15 |

### Example-8

The effects of weight ratios of lornoxicam to alkalinizer(s) on lornoxicam dissolution were studied. The different compositions are shown in table no. 10 & 12 and the results are shown in table 11 and 13.

TABLE 10

| S. | | 1798/021 | 1798/043 | 1798/039 | 1798/061 | 1798/063 |
|---|---|---|---|---|---|---|
| No. | Ingredients (%) | (1:37.5) | (1:39.5) | (1:40) | (1:43) | (1:44) |
| 1 | Lornoxicam | 1.00 | 1.00 | 1.00 | 1.00 | 1.00 |
| 2 | MCC (Avicel | 49.75 | 47.75 | 47.25 | 44.25 | 43.25 |
| | PH102) | | | | | |
| 3 | Sodium | 37.50 | 39.50 | 40.00 | 43.00 | 44.00 |
| | Bicarbonate | | | | | |
| 4 | Fumaric acid | 6.25 | 6.25 | 6.25 | 6.25 | 6.25 |
| 5 | Sodium starch | 5.00 | 5.00 | 5.00 | 5.00 | 5.00 |
| | glycollate | | | | | |
| 6 | Magnesium stearate | 0.50 | 0.50 | 0.50 | 0.50 | 0.50 |
| | TOTAL | 100.00 | 100.00 | 100.00 | 100.00 | 100.00 |

TABLE 11

| | % Dissolution (% release) | | | | |
|---|---|---|---|---|---|
| Time | 1798/021 | 1798/043 | 1798/039 | 1798/061 | 1798/063 |
| (Min.) | (1:37.5) | (1:39.5) | (1:40) | (1:43) | (1:44) |
| 1 | 14.0 | 25.0 | 18.0 | 20.0 | 37.0 |
| 2 | 17.0 | 30.0 | 34.0 | 39.0 | 45.0 |
| 3 | 20.0 | 30.0 | 34.0 | 43.0 | 46.0 |
| 4 | 20.0 | 30.0 | 32.0 | 42.0 | 47.0 |
| 5 | 21.0 | 28.0 | 30.0 | 41.0 | 46.0 |
| 10 | 26.0 | 27.0 | 25.0 | 42.0 | 46.0 |
| 15 | 28.0 | 28.0 | 29.0 | 40.0 | 45.0 |
| 20 | 38.0 | 31.0 | 35.0 | 52.0 | 60.0 |

TABLE 12

| | Ingredients | 1798/053 | 1798/055 | 1798/041 | 1798/045 |
|---|---|---|---|---|---|
| S. No. | (%) | (1:45) | (1:50) | (1:85) | (1:85.5) |
| 1 | Lornoxicam | 1.00 | 1.00 | 1.00 | 1.00 |
| 2 | MCC (Avicel | 42.25 | 37.25 | 2.25 | 1.74 |
| | PH102) | | | | |
| 3 | Sodium | 45.00 | 50.00 | 85.00 | 85.50 |
| | Bicarbonate | | | | |
| 4 | Fumaric acid | 6.25 | 6.25 | 6.25 | 6.25 |
| 5 | Sodium starch | 5.00 | 5.00 | 5.00 | 5.00 |
| | glycollate | | | | |
| 6 | Magnesium | 0.50 | 0.50 | 0.50 | 0.50 |
| | stearate | | | | |
| | TOTAL | 100.00 | 100.00 | 100.00 | 100.00 |

TABLE 13

| | % Dissolution (% release) | | | |
|---|---|---|---|---|
| Time | 1798/053 | 1798/055 | 1798/041 | 1798/045 |
| (Min.) | (1:45) | (1:50) | (1:85) | (1:85.5) |
| 1 | 20.0 | 17.0 | 50.0 | Tablet capping |
| 2 | 56.0 | 51.0 | 51.0 | problem |
| 3 | 46.0 | 54.0 | 53.0 | observed during |
| 4 | 48.0 | 55.0 | 56.0 | compression. |
| 5 | 44.0 | 55.0 | 56.0 | |
| 10 | 42.0 | 56.0 | 62.0 | |
| 15 | 40.0 | 57.0 | 70.0 | |
| 20 | 46.0 | 68.0 | 84.0 | |

The result as shown in tables 11, 12 and 13 clearly indicate that in order to get the desired dissolution profile, the weight ratio of lornoxicam to alkalinizer(s) should be in the range of 1:43 to 1:85

### Example-9

The effects of weight ratios of alkalinizer(s) to organic acids on lornoxicam dissolution were studied. The different compositions are shown in table no. 14 & 16 and the results are shown in table 15 and 17.

TABLE 14

| S. | | 1798/023 | 1798/047 | 1798/067 | 1798/065 | 1798/057 | 1798/059 |
|---|---|---|---|---|---|---|---|
| No. | Ingredients (%) | (2.85:1) | (3:1) | (4:1) | (4.5:1) | (5:1) | (7:1) |
| 1 | Lornoxicam | 1.00 | 1.00 | 1.00 | 1.00 | 1.00 | 1.00 |
| 2 | MCC (Avicel | 9.13 | 10.17 | 15.36 | 17.11 | 18.50 | 22.07 |
| | PH102) | | | | | | |
| 3 | Sodium | 62.50 | 62.50 | 62.50 | 62.50 | 62.50 | 62.50 |
| | Bicarbonate | | | | | | |
| 4 | Fumaric acid | 21.87 | 20.83 | 15.63 | 13.89 | 12.50 | 8.93 |
| 5 | Sodium starch | 5.00 | 5.00 | 5.00 | 5.00 | 5.00 | 5.00 |
| | glycollate | | | | | | |
| 6 | Magnesium | 0.50 | 0.50 | 0.50 | 0.50 | 0.50 | 0.50 |
| | stearate | | | | | | |
| | TOTAL | 100.00 | 100.00 | 100.00 | 100.00 | 100.00 | 100.00 |

TABLE 15

| | % Dissolution (% release) | | | | | |
|---|---|---|---|---|---|---|
| Time | 1798/023 | 1798/047 | 1798/067 | 1798/065 | 1798/057 | 1798/059 |
| (Min.) | (2.85:1) | (3:1) | (4:1) | (4.5:1) | (5:1) | (7:1) |
| 1 | 30.0 | 23.0 | 49.0 | 32.0 | 21.0 | 37.0 |
| 2 | 33.0 | 39.0 | 58.0 | 45.0 | 43.0 | 45.0 |
| 3 | 34.0 | 38.0 | 58.0 | 46.0 | 49.0 | 52.0 |
| 4 | 33.0 | 41.0 | 55.0 | 49.0 | 48.0 | 52.0 |
| 5 | 33.0 | 39.0 | 58.0 | 50.0 | 49.0 | 49.0 |
| 10 | 32.0 | 37.0 | 59.0 | 52.0 | 49.0 | 50.0 |
| 15 | 31.0 | 37.0 | 57.0 | 53.0 | 51.0 | 57.0 |
| 20 | 34.0 | 38.0 | 63.0 | 75.0 | 81.0 | 81.0 |

TABLE 16

| S. | | 1798/49 | 1798/051 | 1798/025 | 1798/071 | 1798/069 |
|---|---|---|---|---|---|---|
| No. | Ingredients (%) | (20:1) | (20.5:1) | (25:1) | (50:1) | (100:1) |
| 1 | Lornoxicam | 1.00 | 1.00 | 1.00 | 1.00 | 1.00 |
| 2 | MCC (Avicel | 27.88 | 27.95 | 28.50 | 29.75 | 30.36 |
| | PH102) | | | | | |
| 3 | Sodium | 62.50 | 62.50 | 62.50 | 62.50 | 62.50 |
| | Bicarbonate | | | | | |
| 4 | Fumaric acid | 3.12 | 3.05 | 2.50 | 1.25 | 0.63 |
| 5 | Sodium starch | 5.00 | 5.00 | 5.00 | 5.00 | 5.00 |
| | glycollate | | | | | |
| 6 | Magnesium | 0.50 | 0.50 | 0.50 | 0.50 | 0.50 |
| | stearate | | | | | |
| | TOTAL | 100.00 | 100.00 | 100.00 | 100.00 | 100.00 |

TABLE 17

| | % Dissolution (% release) | | | | |
|---|---|---|---|---|---|
| Time | 1798/49 | 1798/051 | 1798/025 | 1798/071 | 1798/069 |
| (Min.) | (20:1) | (20.5:1) | (25:1) | (50:1) | (100:1) |
| 1 | 23.0 | 22.0 | 15.0 | 19.0 | 14.0 |
| 2 | 39.0 | 38.0 | 34.0 | 36.0 | 19.0 |
| 3 | 38.0 | 45.0 | 40.0 | 43.0 | 37.0 |
| 4 | 41.0 | 45.0 | 42.0 | 45.0 | 39.0 |
| 5 | 39.0 | 47.0 | 44.0 | 47.0 | 43.0 |
| 10 | 37.0 | 52.0 | 52.0 | 52.0 | 57.0 |
| 15 | 37.0 | 56.0 | 57.0 | 61.0 | 53.0 |
| 20 | 38.0 | 90.0 | 88.0 | 81.0 | 91.0 |

The result as shown in tables 15 and 17 clearly indicate that in order to get the desired dissolution profile, the weight ratio of alkalinizer(s) to organic acid should be in the range of 3:1 to 100:1

While considerable emphasis has been placed herein on the specific ingredients of the preferred composition, it will be appreciated that many additional ingredients can be added and that many changes can be made in the preferred composition without departing from the principles of the invention. These and other changes in the preferred composition of the invention will be apparent to those skilled in the art from the disclosure herein, whereby it is to be distinctly understood that the foregoing descriptive matter is to be interpreted merely as illustrative of the invention and not as a limitation.

1. A fast dissolving pharmaceutical composition comprising:
a. lornoxicam or pharmaceutically acceptable salts thereof;
b. at least one alkalinizer;
c. at least one organic acid; and
d. pharmaceutically acceptable excipients,
wherein the weight ratio of lornoxicam to alkalinizer(s) is in the range of from about 1:43 to about 1:85 and the weight ratio of alkalinizer(s) to organic acid(s) is in the range of from about 3:1 to about 100:1,
wherein at least 30% of lornoxicam is released from said composition within 3 minutes and at least 40% of lornoxicam is released from said composition within 10 minutes.

2. The composition as claimed in claim 1, wherein said composition exhibits a lornoxicam plasma Tₘₐₓ of less than 45 minutes.

3. The composition as claimed in claim 1, wherein said composition exhibits a lornoxicam plasma Cₘₐₓ of more than 950 ng/ml after the administration of a 8 mg dose of lornoxicam.

4. The composition as claimed in claim 1, wherein said composition exhibits a lornoxicam AUC_{(0-10 min)} of about 10 ng.h/ml to about 70 ng.h/ml after the administration of a 8 mg dose of lornoxicam.

5. The composition as claimed in claim 1, wherein said composition exhibits a lornoxicam AUC_{(0-20 min)} of about 70 ng.h/ml to about 200 ng.h/ml. after the administration of a 8 mg dose of lornoxicam

6. The composition as claimed in claim 1, wherein said composition exhibits a lornoxicam AUC_{(0-30 min)} of about 200 ng.h/ml to about 500 ng.h/ml after the administration of a 8 mg dose of lornoxicam.

7. The composition as claimed in claim 1, wherein the amount of lornoxicam or pharmaceutically acceptable salts thereof is in the range of about 0.5% to about 2.5% of the mass of the composition.

8. The composition as claimed in claim 1, wherein the alkalinizer is at least one selected from the group comprising sodium bicarbonate, potassium bicarbonate, calcium carbonate, sodium carbonate and combinations thereof.

9. The composition as claimed in claim 1, wherein the alkalinizer is a bicarbonate.

10. The composition as claimed in claim 1, wherein the alkalinizer is a mixture of bicarbonate and carbonate.

11. The composition as claimed in claim 1, wherein the amount of alkalinizer is in the range of about 43% to about 85% of the mass of the composition.

12. The composition as claimed in claim 1, wherein the organic acid is at least one selected from the group comprising fumaric acid, citric acid, tartaric acid, succinic acid, glycine and combinations thereof.

13. The composition as claimed in claim 1, wherein the amount of the organic acid is in the range of about 3% to about 20% of the mass of the composition.

14. The composition as claimed in claim 1, further comprises at least one water uptake agent selected from the group comprising cross-linked polyvinylpyrrolidone, croscarmellose sodium, sodium starch glycolate, starch, starch derivatives, hydroxypropylcellulose, low substituted hydroxypropylcellulose, hydroxypropylmethyl cellulose, alginic acid, sodium alginate, calcium sulphate, calcium carboxymethylcellulose, microcrystalline cellulose, powdered cellulose, colloidal silicon dioxide, docusate sodium, guar gum, magnesium aluminium silicate, methylcellulose, polarcrilin potassium, silicified microcystalline cellulose, magnesium oxide, tragacanth, mannitol, sorbitol, xylitol, sucrose, lactose, fructose, maltose, polyethylene glycol, amino acids, cyclodextrin, urea, polyvinylpyrrolidone and combinations thereof.

15. The composition as claimed in claim 14, wherein the amount of water uptake agent is in the range of about 20% to about 50% of the mass of the composition.

16. A fast dissolving pharmaceutical composition comprising:
a) lornoxicam or pharmaceutically acceptable salts thereof in an amount of about 0.5% to about 2.5% of the mass of the composition;
b) at least one alkalinizer;
c) at least one organic acid; and
d) pharmaceutically acceptable excipients
wherein the weight ratio of lornoxicam to alkalinizer(s) is in the range of from about 1:43 to about 1:85 and the weight ratio of alkalinizer(s) to organic acid(s) is in the range of from about 3:1 to about 100:1;
wherein said composition exhibits a dissolution profile within the following ranges:

| Time | lornoxicam |
|---|---|
| 3 minutes | at least 30% |
| 10 minutes | at least 40% |

in a USP type II apparatus at 30 rpm using 0.0033 M HCl as a dissolution medium at 37±0.5° C.

17. A process for the preparation of a fast dissolving pharmaceutical composition; said process comprising the following steps:
(a) mixing lornoxicam or pharmaceutically acceptable salts thereof with at least one alkalinizer and other pharmaceutically acceptable excipients to obtain a mixture;
(b) optionally granulating the mixture;
(c) adding at least one organic acid to the mixture to obtain a blend,
(c) compressing the blend to form a tablet; and
(d) optionally coating the tablet to convert it into pharmaceutically acceptable film coated tablet;
wherein the weight ratio of lornoxicam:alkalinizer(s) is in the range of from about 1:43 to about 1:85 and the weight ratio of alkalinizer(s):organic acid(s) is in the range of from about 3:1 to about 100:1.
